(19) United States
(12) Patent Application Publication
(10) Pub. No.: US 2013/0144211 A1
(43) Pub. Date: Jun. 6, 2013

(54) ADAPTER FOR DRILL DRIVE AND LAVAGE SYSTEM

(71) Applicant: Heraeus Medical GmbH, Wehrheim (DE)

(72) Inventors: Sebastian Vogt, Erfurt (DE); Marie Katharina Apitius, Remagen (DE)

(73) Assignee: HERAEUS MEDICAL GMBH, Wehrheim (DE)

(21) Appl. No.: 13/705,366

(22) Filed: Dec. 5, 2012

(30) Foreign Application Priority Data
Dec. 5, 2011 (DE) 10 2011 120 087.1
Jan. 12, 2012 (DE) 10 2012 000 392.7

(51) Int. Cl. A61M 3/02

(52) U.S. Cl. CPC A61M 3/02
USPC 604/131

(57) ABSTRACT

Adapter for drill driven lavage system with first fastener for connecting the adapter to a drill drive, and second fastener for connecting the lavage system to the adapter. A shaft supported to rotate in axial direction and to connect to the drill drive that the shaft can be rotated by the drill drive. The adapter comprises a disc which is connected to the shaft in such manner that the disc rotates along when the shaft rotates. The disc comprises an attachment in an eccentric arrangement or the disc is inclined towards the rotation axis of the shaft. The adapter is connected to the lavage system by second fastener so that the rotating disc drives the lavage system. The adapter is connected to the lavage system by means of the second fastening facility in such manner that the pestle drives the lavage system and hits periodically onto a membrane of the lavage system.

This application for patent claims priority to DE 10 2011 120 037.1 filed on Dec. 5, 2012 and DE 10 2012 000 392.7 of Jan. 12, 2012.

### BACKGROUND OF THE INVENTION

The invention relates to an adapter for a drill drive and a lavage system as well as a drill drive and a lavage system having an adapter.

Lavage systems are used during bone surgery such as, for example, the insertion of an artificial hip joint, to gently, but effectively, rinse away residues generated during the operation in order to prevent complications from arising after the operation and to accelerate healing. The residues can adversely affect the durability of an artificial hip joint and lead to bone infection.

For this purpose, the lavage system produces a pulsating jet of a liquid. In most cases, the liquid is sterile water and/or an aqueous solution that also contains pharmaceutically effective substances. The rinsing water containing the residues is aspirated by the lavage system through a second channel.

Lavage systems (also called jet lavages) comprise a membrane pump that is used to generate the pulsating jet of liquid and to convey the liquid. Prior to its use, a drive module is connected to the lavage system and serves to drive the membrane pump.

A lavage system is known from DE 10 2010 046 057.5, a document that was not pre-published, in which a linear oscillating pestle is driven by means of a compressed gas motor. The pestle periodically hits onto a membrane of the lavage system and thus forms a membrane pump in periodical operation for generating a pulsating jet of liquid.

This is disadvantageous in that compressed air or a compressed gas needs to be supplied to the motor. For this purpose, the compressed gas motor needs to either be connected to a compressed gas line or comprise a compressor having electrical cables or a compressed gas cartridge.

US 2003 036 723 A1 discloses a lavage system having a pump that is driven by means of an electrical motor via a gear. Said pump generates the jet of liquid. The energy for the motor is supplied through an electrical cable.

The complex structure of said lavage system is disadvantageous. Due to the hygienic requirements, at least parts of the lavage system need to be sterilisable or the lavage system needs to be a disposable product. This is expensive or very resource-consuming in the lavage system according to US 2003 036 723 A1.

DE 10 2009 021 421 A1 proposes a jet lavage having a lifting magnet driving the membrane pump. Said disposable product has a significantly less expensive structure already.

It is disadvantageous that the use of a lifting magnet also generates costs and consumes resources. The elements required to build up magnets have become expensive lately. Magnetisable materials for production parts have to be avoided in the design of devices having magnets. Accordingly, the design is still resource-consuming and expensive.

### BRIEF SUMMARY OF THE INVENTION

It is the object of the present invention to overcome these and other disadvantages that have not been specified above. In particular, a less expensive system is to be provided that is easy to insert and can be designed to be resource-saving. Preferably, existing lavage systems should still be usable.

The objects are met through an adapter for a drill drive for driving a lavage system comprising a first fastening facility for connecting the adapter to the drill drive, a second fastening facility for connecting the lavage system to the adapter, and a shaft supported like in a bearing such that it can be rotated in axial direction and can be connected to the drill drive at a first end of the shaft in such manner that the shaft can be rotated by means of the drill drive, whereby the adapter comprises a disc that is connected to the shaft at a second end of the shaft opposite from the first end in such manner that the disc rotates along when the shaft rotates, whereby the disc comprises, on the side facing away from the shaft, an attachment or a projection in an eccentric arrangement and/or the disc is inclined towards the rotation axis of the shaft, whereby the adapter can be connected to the lavage system by means of the second fastening facility in such manner that the rotating disc drives the lavage system on the side facing away from the shaft or via the attachment or the projection.

In the scope of the present invention, the shaft can comprise the first fastening facility. For this purpose, a part of the shaft serving as first fastening facility can be taken up, for example, into a drill chuck of a drill drive. Together with the part of the shaft taken up, the tightened drill chuck forms a fastening that connects the adapter to the drill drive and simultaneously provides the drive for the shaft. For this purpose, according to the invention, the shaft can comprise a pin and/or one or more flat parts that provide for better grip to the drill drive.

In the scope of the invention, the term, disc, shall be interpreted broadly. According to the invention, a disc can, in an extreme case, be a rod having an essentially cylindrical cross-section. However, circular discs are particularly preferred to embody the present invention, since they can be rotated without any major imbalance.

The invention can provide, in case of the adapters, the attachment or the projection or the rotating disc, on the side facing away from the shaft, to press via a pressure point directly onto a membrane of the lavage system, whereby the pressure point runs over the membrane periodically when the disc rotates.

The structure is particularly easy and inexpensive to implement since only one moving part (shaft with disc and attachment) needs to be incorporated into the adapter.

Alternatively, the invention can provide the attachment or the projection or the disc to have a piston rod connected to it by means of a connection at a first end of the piston rod, whereby the connection can be rotated about at least one axis, such that a second end of the piston rod performs a periodical hitting motion when the disc rotates, whereby the second end of the piston rod preferably drives the lavage system, provided a lavage system is connected, whereby it is particularly preferred for the second end of the piston rod to periodically hit onto a membrane of the lavage system, provided a lavage system is connected.

Although an adapter of this type has a more resource-consuming structure, it offers improved pumping performance due to the piston rod.

Another alternative refinement of the invention can provide the adapter to comprise a plunger that is mobile along the plunger axis and elastically supported like in a bearing, whereby the plunger can be deflected periodically in the direction of the plunger axis through the rotating disc and can be restored by means of being supported elastically like in a bearing, whereby the elastic support preferably is a spring, in particular a steel spring.

Said adapter requires a spring and two moving parts, but needs no joints. The still simple structure and the good pumping performance due to the use of a linearly mobile plunger renders said adapters particularly preferred according to the invention.

The embodiment can provide the plunger to comprise a slanted surface on the side towards the disc and/or the plunger to be supported like in a bearing in the adapter such as to be torque-proof about the plunger axis.

Due to the plunger side facing towards the disc being slanted, a periodical linear motion of the plunger as a pestle for a membrane pump of a lavage system upon rotation of the disc is attained.

Moreover, the invention can provide the plunger end situated opposite from the disc, which is preferably rounded, to drive the lavage system, provided a lavage system is connected, in particular provide the plunger to periodically hit onto the membrane of the lavage system, provided a lavage system is connected, when the disc rotates.

The purpose of the rounding of the plunger is to prevent the membrane from being damaged by corners and edges of the plunger.

Preferably, adapters according to the invention can be provided such that the disc is an eccentric tappet, whereby the region of the eccentric tappet that is farthest from the shaft preferably drives the lavage system, provided a lavage system is connected, in particular such that the region of the rotating eccentric tappet that is farthest from the rotating shaft periodically hits onto the membrane of the lavage system, provided a lavage system is connected.

The objects of the invention are also met through an adapter for a drill drive for driving a lavage system, comprising a first fastening facility for connecting the adapter to the drill drive, a second fastening facility for connecting the lavage system to the adapter, and a shaft supported like in a bearing such that it can be rotated axially that can be connected to the drill drive at a first end of the shaft in such manner that the shaft can be rotated by means of the drill drive, whereby the shaft comprises a crankshaft that drives a pestle, whereby the adapter can be connected to the lavage system by means of the second fastening facility in such manner that the pestle drives the lavage system, and whereby it is preferred for the pestle to periodically hit onto the membrane of the lavage system.

Although the use of a crankshaft leads to an imbalance, said structure is particularly simple. The bearing of the crankshaft must be sturdier than for the use of balanced shafts and discs.

The invention can provide in all adapters according to the invention for the first fastening facility to be implemented at the first end of the shaft, whereby the first fastening facility preferably is a quick-lock coupling part for a quick-lock coupling of the drill drive and/or the shaft comprises at least one surface, at least one recess and/or at least one pin at the first end of the shaft through which a form-fitting connection to the drill drive can be or is established by means of an opposite snap-in locking means of the drill drive.

Since the torque of the drill drive needs to be transferred to the shaft, it is sufficient to have a first fastening facility at the shaft which is therefore a particularly simple embodiment of the invention.

Moreover, the invention can provide for all adapters according to the invention that the second fastening facility is a bayonet lock through which the lavage system can be or is connected to the adapter.

According to another refinement, the invention can provide the shaft to be a flexible shaft or a joint shaft, in particular a constant velocity joint shaft, comprising at least one joint.

This allows the linear periodical motion driving the membrane pump to not have to be in the direction of the rotation axis of the drill drive. This allows user-friendly lavage systems with a drill drive to be designed.

The invention can just as well provide the adapter to comprise a housing that surrounds the shaft and preferably the disc also, whereby the adapter, in particular, is fully enclosed through the housing except for the region of the fastening facilities such that the connected adapter preferably is enclosed with respect to the outside through the housing.

Having the housing prevents malfunctions and secures a source of hazard, namely rapid rotating components.

The objects of the invention are also met through a drill drive comprising an adapter of said type, in which the shaft of the adapter can be or is fastened in a fastening means, in particular in a drill chuck of the drill drive, and the shaft can be or is driven by the drill drive.

In this context, the invention can provide the drill drive to comprise an electrical motor, a rechargeable battery or a battery, at least one operating facility, and a handle, whereby the rechargeable battery or the battery is connected to the electrical motor and the rechargeable battery or the battery can supply electrical energy to the electrical motor, whereby the electrical motor can be controlled through the operating facility.

The objects of the invention are also met through a lavage system comprising an adapter of said type or comprising a drill drive of said type, in which the lavage system is connected to the adapter in appropriate manner such that the rotating disc, preferably the attachment, the projection or the eccentric tappet or a pestle performing a periodical linear motion through the action of the rotating disc or of a crankshaft, preferably the piston rod or the plunger, drive the lavage system.

In this context, the invention can provide the lavage system to comprise a flexible membrane onto which the adapter acts through a periodical motion when the shaft rotates, preferably by means of periodical hits of the pestle that is moved in linear direction.

Moreover, the invention can provide the periodical drive by means of the adapter to effect periodical spray puffs of the lavage system, whereby the lavage system can be or is connected to a liquid reservoir, whereby the lavage system and the adapter form a pumping facility of the lavage system that can be used to pump liquid from the liquid reservoir into the lavage system.
The invention is based on the surprising finding that an adapter with a simple design can be used to combine drill drives, which are usually present in operating theatres anyway, and simple lavage systems in order to thus provide an inexpensive design of a lavage system having a drive. For this purpose, the adapter converts the rotating motion of the drill drive into a periodical motion that acts on the membrane of an existing lavage system and thus forms a membrane pump that is used to generate the periodical liquid jet. For this purpose, according to the invention, the adapter comprises a fastening facility for fastening the lavage system to the adapter and a fastening facility for fastening the drill drive to the adapter. Accordingly, the adapter serves as an intermediate piece for a drill drive, which is usually present in operating theatres, and an inexpensive lavage system attachment and/or lavage system.

The various embodiments of the invention are simple implementation variants reflecting the scope of the invention. The design of the adapter remains quite simple in all cases. Moreover, the adapter can be used repeatedly whereas only a lavage attachment or a lavage annex, herein also referred to as lavage system, needs to be sterilised and/or replaced.

### BRIEF DESCRIPTION OF THE DRAWINGS

Exemplary embodiments of the invention shall be illustrated in the following on the basis of six schematic figures, though without limiting the scope of the invention. In the figures:

FIG. 1: shows a schematic cross-sectional view of a lavage system according to the invention having an adapter according to the invention and a drill drive;

FIG. 2: shows a schematic cross-sectional view of an adapter according to the invention having a plunger supported in elastic manner;

FIG. 3: shows a schematic top view onto an adapter according to the invention having a joint shaft and a pestle with the housing being open;

FIG. 4: shows a schematic top view onto an adapter according to the invention having an eccentric tappet and a pestle;

FIG. 5: shows a schematic cross-sectional view of an adapter according to the invention having an inclined disc and a slanted plunger; and

FIG. 6: shows a schematic cross-sectional view of an adapter according to the invention having a joint shaft and a pestle with a ball joint.

### DETAILED DESCRIPTION OF THE INVENTION

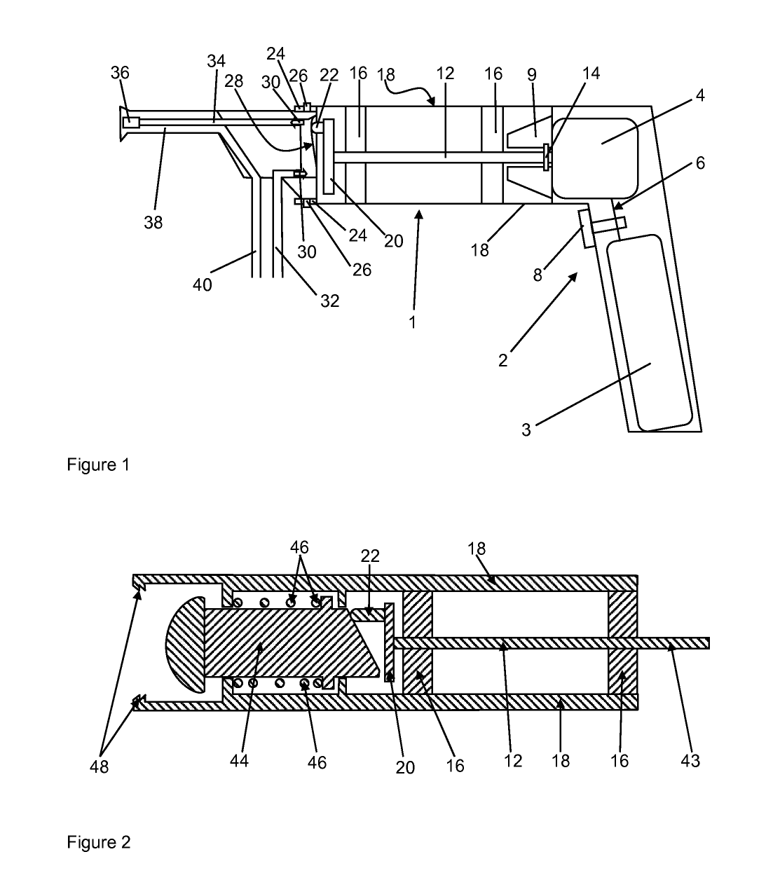

FIG. 1 shows a schematic cross-sectional view of a lavage system according to the invention having a lavage attachment, adapter 1, and drill drive 2. The drill drive 2 comprises a rechargeable battery 3 as energy source and an electrical motor 4 that provides the torque for the drill drive 2. The rechargeable battery 3 is arranged in a handle of the drill drive 2. The electrical motor 4 is connected to the rechargeable battery 3 through an electrical cable 6. The electrical motor 4 can be switched on and off by means of an operating facility 8 in the form of a switch 8. It is also conceivable to control the rotary speed of the electrical motor 4 through the operating facility 8.

A drill chuck 9 or any other fastening facility is situated on the front of the drill drive 2. A shaft 12 of the adapter 1 is clamped into said drill chuck 9. The shaft 12 comprises a pin 14 which also serves for form-fitting fastening of the shaft 12 to the drill drive 2. The first end of the shaft 12 having the pin 14 is the first fastener according to the scope of the present invention.

Two brackets 16 ensure that the shaft 12 is supported like in a bearing such as to be fixed in position with respect to the brackets 16, but capable of rotating in brackets 16. The brackets 16 are connected to a housing 18 that surrounds the connected adapter 1.

A disc 20 is arranged in the adapter 1 at the second end of the shaft 12, opposite the first end of the shaft 12. The disc 20 is circular and the axis of the shaft 12 coincides with the centre of the circular disc of disc 20. An attachment 22 is arranged on the disc 20 in eccentric position such that the attachment rotates in a circle when the disc 20 rotates.

A bayonet lock 24 is arranged at the front of the adapter 1 and engages short pins 26 of the lavage attachment such that the lavage attachment is connected to the adapter 1 in detachable manner. The lavage system comprises a flexible membrane 28 onto which the attachment 22 of disc 20 presses when the adapter 1 is connected to the lavage attachment, as shown. Accordingly, the attachment 22 presses into the membrane 28 in the position shown. The flexible membrane 28 can be made, for example, from gum elastic. If the disc 20 is inclined slightly with respect to the axis of the shaft 12, the attachment 22 periodically hits against the membrane 28 when the disc 20 rotates.

The membrane 28 forms a wall of a space that is connected to two lines 32, 34 through first and second valves 30. The first line 32 feeds a liquid to the space, whereas the second line 34 guides the liquid to a nozzle 36 through which the liquid can exit.

Subsequently, the liquid, which is then mixed with residues, can be aspirated again through a suction line 38 that surrounds the second line 34 in a coaxial manner. Subsequently, the liquid can be removed from the lavage system through a discharge line 40.

The second valve 30 in the first line 32 blocks the liquid from exiting the space and entering the first line 32 and allows liquid to enter the space. The first valve 30 in the second line 34 blocks liquid from entering the space from the second line 34 and allows liquid to exit from the space and entering the second line 34. The space having the membrane 28 thus forms a membrane pump that is driven through the attachment 22 running periodically over the membrane 28.

The volume of the space is reduced when the attachment 22 presses into the membrane 28. Then, the liquid is pushed from the space into the second line 34 and is ejected from the nozzle 36 as a spray puff. When the attachment 22 migrates to a position in which it does not press onto the membrane 28 or in which the membrane 28 changes shape less strongly, the volume of the space increases. This aspirates liquid from the first line 32 through the lower valve 30 into the space.

With ongoing rotation of the disc 20 and/or ongoing rotation of the shaft 12 and thus the membrane 28 being impressed periodically through the attachment 22, periodical spray puffs of the liquid exit from the nozzle 36.

All parts of the adapter 1 can consist of plastic materials and can be manufactured through injection moulding. An imbalance of disc 20 caused through the attachment 22 can be balanced through a counter-weight (not shown) on the opposite side or on the external edge of the disc 20.

FIGS. 2 to 6 each show only the adapters according to the invention though these can be readily connected to lavage attachments and/or drill drives like the ones shown in FIG. 1, in order to form drill drives according to the invention or lavage systems according to the invention.

FIG. 2 shows a schematic cross-sectional view of an alternative adapter having a central shaft 12 that is supported like in a bearing such that it can be rotated. A first end 43 of the shaft 12 serves as fastening means for fastening a drill drive (not shown). For this purpose, the first end 43 of the shaft 12 can be clamped into the drill drive.

The shaft 12 is supported like in a bearing in two brackets 16 such that it can be rotated. The brackets 16 are connected to the housing 18 of the adapter in fixed manner. The housing 18 has a cylindrical shape towards the outside. A disc 20 is arranged at the end of the shaft 12 that is opposite to the first end 43, whereby the disc 20 can take any shape as long as it can be rotated freely about the axis of the shaft 12 inside the housing 18. The disc 20 could just as well be implemented in the form of a kinked shaft (not shown).

An attachment 22 is arranged on said disc 20. However, the disc 20 could just as well be deformed-at the respective site or the kinked shaft has another kink. These embodiments are refinements according to the invention of the adapter as well.

The attachment 22 presses onto a linearly mobile plunger 44 that is supported like in a bearing on the inside of the housing 18. The plunger 44 comprises a mushroom-shaped head intended to periodically hit a membrane of a lavage system that includes the adapter (not shown).

A metal spring 46 presses the plunger 44 in the direction of the disc 20 and thus against the attachment 22. The plunger 44 has a rectangular cross-section and is positioned in rectangular recesses in the housing 18. This ensures that the plunger 44 is supported like in a bearing such that it cannot be rotated. Any other geometry of the plunger 44 and/or of the recesses in the housing 18, in which the plunger 44 is supported like in a bearing and which prevent the plunger 44 from rotating, fulfil the same purpose according to the invention.

The plunger 44 is slanted on the side of the plunger 44 facing the disc 20. When the shaft 12 rotates, the attachment 22 sweeps over the slanted end of the plunger 44, which deflects the plunger 44 periodically. The adapter thus generates periodical hits of the plunger 44 from the rotation of the shaft 12.

At the second end of the adapter opposite the first end 43, the inside of the housing 18 has a snap-in locking facility 48 arranged in it that is designed to engage an opposite snap-in locking facility of a lavage attachment (not shown) such that a lavage system can be built up easily from the lavage attachment and the adapter.

With the exception of the spring 46, all parts of the adapter are made of plastic materials and are manufactured through injection moulding. As a matter of principle, even the spring 46 could be manufactured from an elastic plastic material.

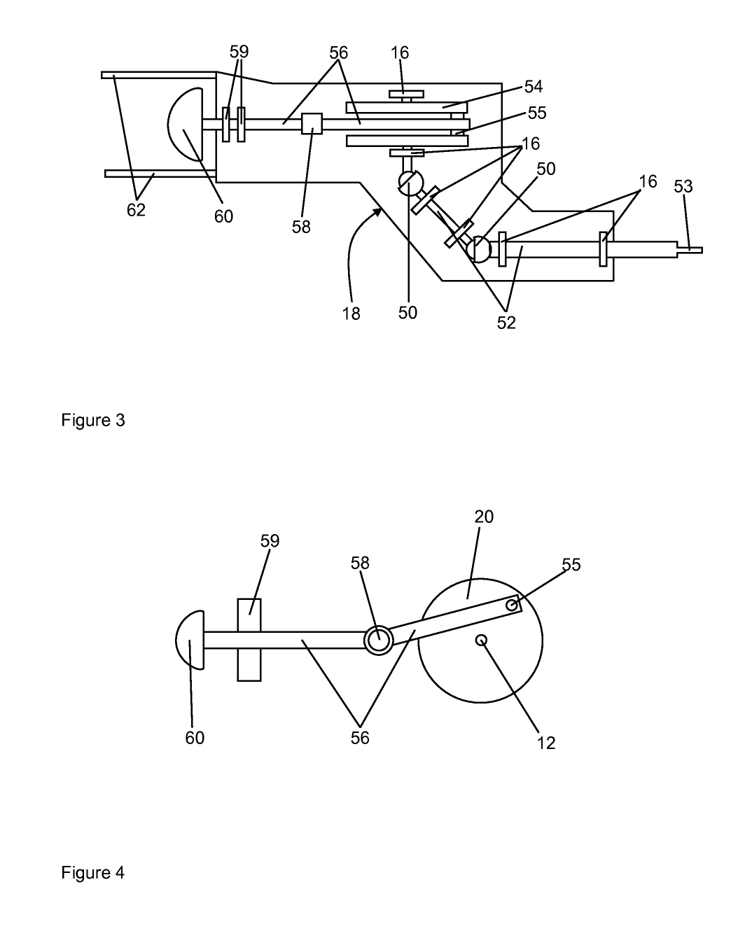

FIG. 3 shows a schematic top view onto another adapter according to the invention with the housing 18 being open. This top view allows the structure inside the housing 18 to be seen well. The adapter comprises a joint shaft 52 whose rotation axis is reversed through two ball joints 50. A first end of the joint shaft 52 has a flattened part 53 of the joint shaft 52 provided on it as first fastener through which the adapter can be connected to a drill drive (not shown).

The joint shaft 52 can, for example, be a constant velocity joint shaft or a joint shaft of a simple design. The location of the parts of the joint shaft 52 in the adapter is defined through brackets 16 that are fixedly connected to the housing 18.

The brackets 16 also define the positions of a crankshaft 54 that is connected to the joint shaft 52 such that it can be rotated. The crankshaft 54 is connected by a rotation axis 55 to a pestle 56 that converts the rotary motion of the joint shaft 52 and crankshaft 54 into a linear oscillating motion. For this purpose, the pestle 56 comprises a rotary joint 58 and is supported like in a bearing through two brackets 59 such that it can be shifted linearly. Accordingly, whereas the brackets 16 of the joint shaft 52 and crankshaft 54 enable a rotation in the bracket 16, the brackets 59 are designed such as to enable the pestle 56 to be shifted linearly through the brackets 59. Accordingly, both brackets 16, 59 are not fixed connections to the joint shaft 52 and/or the pestle 56.

In place of a crankshaft 54, a disc or a pair of discs in a parallel arrangement could be supported like in a bearing in the adapter and could be connected to the joint shaft 52. In this case, the pestle 56 is connected in eccentric position to the disc or between the discs by means of an axis.

The side of the pestle 56 to which a lavage attachment can be fastened terminates in a rounded head 60. The housing 18 is lengthened in the region of the head 60 and forms a second fastening facility 62 in this location for a lavage system (not shown) that can be plugged into the second fastening facility 62.

Building a lavage system with adapter through attaching a lavage system to the second fastening facility 62 and fastening a drill drive to the joint shaft 52 on the adapter, driving the joint shaft 52 moves the pestle 56 in a periodically linear oscillating manner such that the head 60 periodically hits onto a membrane of the lavage system and thus operates a membrane pump that effects periodical spray puffs of the lavage system.

The exclusive purpose of the joint shaft 52 is to ensure that the linear motion of the pestle 56 and thus of the head 60 occurs in the direction of the rotation axis of the drill drive. The lavage system comprising the drill drive and the attached lavage system can then be used in the manner of a pistol. Alternatively, the attachment, i.e. the lavage system to be attached to the adapter, can be curved appropriately for a joint shaft 52 in the adapter to be dispensable and such that a normal shaft without joints can be used instead.

FIG. 4 shows a schematic view of another alternative embodiment of an adapter according to the invention. A housing (not shown) can be provided to be situated around the adapter. The adapter comprises a disc 20 that is supported like in a bearing such that it can rotate about a shaft 12. The shaft 12 extends further on the backside of the disc 20 (into the image plane in FIG. 4) and terminates at a fastening facility for a drill drive. The shaft 12 forms the rotation axis for the circular disc 20.

A pestle 56 is connected in eccentric manner to the disc 20 by means of a rotation axis 55. The pestle 56 comprises a joint 58 and is supported like in a bearing behind the joint 58 by means of a bracket 59 having a feed-through for the joint arm of the pestle 56 facing away from the disc 20 such that the pestle 56 is capable of performing a linear motion. The pestle 56 terminates behind the bracket 59 in a head 60 that serves as contact site for a membrane.

The hitting direction of the pestle 56 and/or head 60 in this case is perpendicular to the rotation axis of the shaft 12, which can here be provided as a simple rod, and thus perpendicular to the rotation axis of a drill drive provided a drill drive is connected to the adapter. The bracket 59 for the pestle 56 and a bracket for the disc 20, which is situated behind the disc 20 and is therefore not shown in FIG. 4, are connected to each other in fixed manner. The disc 20 is supported like in a bearing in the bracket, which is not shown, such that it can rotate. A fastening facility (not shown) for fastening a lavage system is also connected to the brackets 59 in fixed manner.

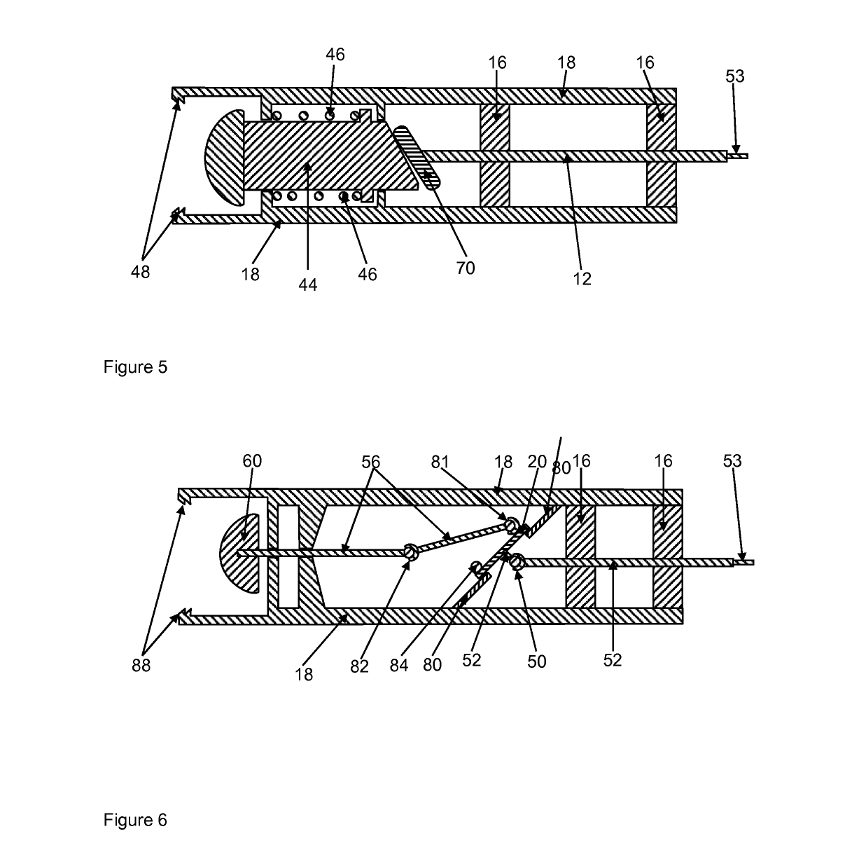

FIG. 5 shows a schematic cross-sectional view of another alternative embodiment of an adapter according to the invention for a lavage system according to the invention or a drill drive according to the invention with a similar design to the one according to FIG. 2. In place of a disc 20 with an attachment 22 according to FIG. 1, the present adapter comprises a disc 70 with rounded edge that is inclined towards the shaft 12. The disc 70 can be circular, rotationally symmetrical and connected to the shaft 12 in the centre of symmetry. However, the disc 70 can just as well be a rod having rounded ends, whereby the rod can have either an angular or a round profile.

Due to the inclination of the disc 70, the front edge of the disc 70 runs over the slanted end of a plunger 44 that is supported like in a bearing in a housing 18 such that it can be shifted linearly. A spring 46 ensures that the plunger 44 is pressed onto the disc 70 regardless of the rotational position of the disc 70. Brackets 16 keep the shaft 12 in position.

Unlike the adapter according to FIG. 2, the present adapter is provided, as fastening facility 53 for a drill drive, with a flattened part 53 of the shaft 12 projecting from the housing 18 and the rear bracket 16 (on the right in FIG. 5). As a matter of principle, the shaft 12 could just as well be implemented through a square-edged or other angle-edged rod which can then be clamped easily into a fastening facility of the drill drive.

A snap-in locking device 48 for fastening a lavage system (not shown) is arranged on the front of the adapter (on the left side in FIG. 5). The functional principle of a lavage system assembled through using the adapter is analogous to the embodiments described above.

In place of an inclined disc 70, the shaft 12 could simply be angled on the left of the left bracket 16. In this case, the left end of the shaft 12 would also run in circular manner over the slanted right surface of the plunger 44 and achieve the same effect as the present disc 70. One disadvantage of this design is the imbalance generated through the asymmetrical shaft 12. If the shaft 12 is made up of lightweight materials or if no high rotation speeds are used, a design of this type would be conceivable though and reflect the scope of the present invention.

FIG. 6 shows a schematic cross-sectional view of another adapter according to the invention. The adapter comprises a joint shaft 52 that is supported like in a bearing in the brackets 16 such that it can rotate. At the rear end (on the right side in FIG. 6), the joint shaft 52 projects from a housing 18, which is connected to the brackets 16 in fixed manner, and has a flattened part 53 in this location for fastening a drill drive. The rotation axis in the joint shaft 52 is reversed by means of a joint 50. The same effect could be achieved with a flexible shaft, which can be used as an alternative according to the invention. The reversed joint shaft 52 is fastened to a disc 20, which is supported in the adapter like in a bearing such that it can be rotated, by means of two brackets 80 that are connected to the housing 18 in fixed manner.

The disc 20 comprises a ball joint 81, which is fastened in eccentric manner and connects a pestle 56 to the disc 20. The pestle 56 comprises another ball joint 82 that subdivides the pestle 56 into two joint arms, whereby, when the disc 20 rotates, the joint arm connected to the disc 20 performs a kind of nutation motion that extends along a conical surface that is shifted as a function of the angle and whose conical tip is formed through the ball joint 82, and whereby the centre of gravity of the joint arm and of the ball joint 82 is shifted sinusoidally in the direction of the rotation axis of the drill drive upon a rotation. The joint arm of the pestle 56 facing away from the disc 20 performs a periodical linear back-and-forth motion and thus drives a head 60 that is fastened to the left end of the pestle 56 and serves as a hitting contact site for hitting onto a membrane of a membrane pump. For this purpose, a lavage system can be connected to the adapter through an internal thread 88 on the housing 18. The left joint arm of the pestle 56 is supported like in a bearing in two brackets 59 such that it can be shifted linearly.

In order to balance any imbalance of the system, the disc 20 has a counter-weight 84 fastened on the side of the disc 20 that is opposite to the ball joint 81. The functional principle of a lavage system assembled including said adapter is analogous to the descriptions provided above.

The features of the invention disclosed in the preceding description and in the claims, figures, and exemplary embodiments, can be essential for the implementation of the various embodiments of the invention both alone and in any combination.

### LIST OF REFERENCE NUMBERS

1 Adapter

2 Drill drive

3 Rechargeable battery

4 Electrical motor

6 Electrical cable

8 Switch/operating facility

9 Drill chuck

12 Shaft

14, 26 Pin

16, 59, 80 Bracket

18 Housing

20, 70 Disc

22 Attachment

24 Bayonet lock

28 Membrane

30 Valve

32, 34 Feed line

36 Nozzle

38, 40 Discharge line

43 First end of the shaft

44 Plunger

46 Spring/bearing

48 Snap-in locking device

50, 81, 82 Ball joint

52 Joint shaft

53 Flattened part

54 Crankshaft

55 Rotation axis

56 Pestle

58 Joint

60 Head

62 Fastening facility

84 Counter-weight

88 Internal thread

What is claimed is:

1. An adapter for a drill drive for driving a lavage system, comprising
a first fastener (14, 26, 53) for connecting the adapter (1) to the drill drive (2);
a second fastener (24, 48, 62, 88) for connecting the lavage system to the adapter (1);
a shaft (12, 52) supported such that the shift is hindered to rotate in axial direction and is connectable to the drill drive (2) at a first end (43) of the shaft (12, 52) such that the drill drive (2) rotates the shaft (12, 52);
wherein the adapter (1) comprises a disc (20, 70) connected to the shaft (12, 52) at a second end of the shaft (12, 52) such that the disc (20, 70) rotates along when the shaft (12, 52) rotates,
wherein the disc (20, 70) comprises, on a side facing away from the shaft (12, 52), an attachment (22) arranged eccentrically,
wherein the adapter (1) is connected to the lavage system by the second fastener (24, 48, 62, 88) such that the rotating disc (20, 70) drives the lavage system on the side facing away from the shaft (12, 52).

2. The adapter according to claim 1, wherein the attachment (22) or the rotating disc (20, 70), on the side facing away from the shaft (12, 52), presses via a pressure point directly onto a membrane (28) of the lavage system, wherein the pressure point runs over the membrane (28) periodically when the disc (20, 70) rotates.

3. The adapter according to claim 1, further comprising
a piston rod (56) connected to the attachment (22) by a connection (55, 81) at a first end of the piston rod (56),
wherein the connection (55, 81) rotates about at least one axis, such that a second end of the piston rod (56) performs a periodical hitting motion when the disc (20, 70) rotates,
wherein the second end of the piston rod (56) drives the lavage system.

4. The adapter according to claim 1, wherein
the adapter (1) comprises a plunger (44) having a plunger axis and being elastically supported,
wherein the plunger (44) is deflected periodically in a direction of the plunger axis through the rotating disc (20, 70) and is restored by an elastic support (46).

5. The adapter according to claim 4, wherein the plunger (44) comprises a slanted surface on the side towards the disc (20, 70).

6. The adapter according to claim 4, wherein the end of the plunger (44) situated opposite from the disc (20, 70), is rounded.

7. The adapter according to claim 1, wherein the disc (20, 70) is an eccentric tappet including a region that is farthest from the rotating shaft (12, 52) which periodically hits onto the membrane (28) of the lavage system.

8. The adapter for a drill drive for driving a lavage system, comprising
a first fastener (14, 26, 53), connecting the adapter (1) to the drill drive (2);
a second fastener (24, 48, 62, 88), connecting the lavage system to the adapter (1); and
a shaft (12, 52) supported such that the shaft is rotatable in axial direction and connected to the drill drive (2) at a first end (43) of the shaft (12, 52) in such manner that the shaft (12, 52) is rotated by the drill drive (2);
wherein the shaft (12, 52) comprises a crankshaft (54) that drives a pestle (56), wherein the adapter (1) is connected to the lavage system by the second fasteners (24, 48, 62, 88) such that the pestle (56) drives the lavage system, and whereby it is preferred for the pestle (56) to periodically hit onto the membrane (28) of the lavage system.

9. The adapter according to claim 8, wherein
the first fastener (14, 26, 53) is implemented at the first end (43) of the shaft (12, 52), wherein the first fastener (14, 26, 53) is a quick-lock coupling part for a quick-lock coupling of the drill drive (2) and/or the shaft (12, 52) which comprises at least one surface (52), at least one recess and/or at least one pin (12) at the first end (43) of the shaft (12, 52) through which a form-fitting connection to the drill drive (2) can be or is established by means of an opposite snap-in locking means of the drill drive (2).

10. The adapter according to claim 8, wherein the second fastening facility (24, 48, 62, 88) is a bayonet lock (24) through which the lavage system is connected to the adapter (1).

11. The adapter according to claim 8, wherein the shaft (12, 52) is a flexible shaft or a joint shaft (52).

12. The adapter according to claim 8, wherein the adapter (1) comprises a housing (18) that surrounds the shaft (12, 52) and the disc (20, 70).

13. A drill drive comprising an adapter according to claim 1, wherein the shaft (12, 52) of the adapter (1) is fastened by a drill chuck (9) of the drill drive (2) and the shaft (12, 52) is driven by the drill drive (2).

14. The drill drive according to claim 13, further comprising an electrical motor (4) connected to at least one of a battery (3) or a rechargeable battery and at least one switch (8), the motor, battery and switch associated with a handle.

15. A lavage system according to claim 1, further comprising a connection to the adapter, such that the rotating disc (20, 70) includes an attachment (22) performing a periodical linear motion through the action of the rotating disc (20, 70) or of a crankshaft (54).

16. The lavage system according to claim 15, wherein the lavage system comprises a flexible membrane (28) onto which the adapter (1) acts through a periodical motion when the shaft (12, 52) rotates.

17. The lavage system according to claim 16, wherein the periodical drive of the adapter (1) effects periodical spray puffs of the lavage system, whereby the lavage system is con nected to a liquid reservoir, wherein the lavage system and the adapter (1) form a pump ing facility of the lavage system.

18. The adapter according to claim 3, wherein the second end of the piston rod (56) drives the lavage system and wherein the second end of the piston rod (56) periodically hits onto the membrane (28) of the lavage system.

19. The adapter according to claim 4, wherein the elastic support (46) is a spring.

20. The adapter according to claim 18, wherein the spring (46) is a steel spring.

21. The adapter according to claim 4, wherein the plunger (44) comprises a slanted surface on the side towards the disc (20, 70) and/or the plunger (44) is supported in the adapter (1) such as to be torque-proof about the plunger axis.

22. The adapter according to claim 4, wherein the end of the plunger (44) situated opposite from the disc (20, 70), is rounded.

23. The adapter according to claim 11, wherein the shaft (12, 52) is a constant velocity joint shaft, comprising at least one joint (50).

24. The adapter according to claim 8, wherein the adapter is fully enclosed by the housing (18) except for the region of the fastening facilities (14, 24, 26, 48, 53, 62, 88) such that the connected adapter (1) is enclosed with respect to the outside through the housing (18).
